(19) United States
(12) Patent Application Publication
(10) Pub. No.: US 2015/0017643 A1
(43) Pub. Date: Jan. 15, 2015

(54) METHOD OF MOLECULAR TYPING

(71) Applicant: NHS Blood & Transplant, Watford (GB)

(72) Inventors: Cristina Navarrete, Watford (GB); Alina Lemnrau, Watford (GB); Winnie Pui Chee Chong, Watford (GB)

(21) Appl. No.: 14/381,630

(22) PCT Filed: Feb. 28, 2013
(86) PCT No.: PCT/GB2013/050504

§ 371 Date: Aug. 28, 2014

(30) Foreign Application Priority Data
Feb. 28, 2012 (GB) 1203485.6

(51) Int. Cl. C12Q 1/68

(52) U.S. Cl. CPC C12Q 1/6827
USPC 435/6.11; 536/23.1

(57) ABSTRACT

The invention relates to the identification of genetic traits in an individual. More specifically, the invention relates to identifying human leukocyte antigen (HLA) alleles and haplotypes in a potential donor sample. The invention can be used for matching a donor with a recipient for transplantation, other medical needs and high-through put screening of samples.

The invention relates to the identification of genetic traits in an individual. More specifically, the invention relates to identifying human leukocyte antigen (HLA) alleles and haplotypes in adult and cord blood haematopoietic stem cell or platelet donors. The invention can be used for matching a donor with a recipient for transplantation or other medical needs. This method may also be used to identify disease and drug susceptible HLA haplotypes in patients and normal individuals. It can also be used to trace the evolution and migration of these haplotypes in certain human populations.

The Human Leucocyte Antigen (HLA) system comprises of a group of highly polymorphic molecules which play a pivotal role in the induction and regulation of the adaptive immune response. Although the main function of the HLA molecules is to present antigenic peptides to the T cells, they have also been shown to be crucial in the acceptance or rejection of transplanted organs, tissues and cells. HLA were also involved in some of the most serious immunological reactions that occur following the transfusion of blood products and were directly and indirectly involved in the susceptibility to a variety of autoimmune and infectious diseases. More recently they have been shown to be involved in the responses to certain drugs.

The genes coding for these antigens were located in very close proximity to each other in the short arm of chromosome 6 and as a result they segregate in strong linkage disequilibrium as a linked haplotype. Due to differences in the expression, structure and function, these genes have been defined as HLA class I and class II.

The HLA polymorphisms were initially defined using serological and cellular techniques but with the development of gene cloning and DNA sequencing, it became possible to perform a detailed analysis of these genes at the single nucleotide level. This analysis has shown the existence of certain locus-specific nucleotide sequences in both coding (exons) and non-coding (introns) regions of the genes and also the existence of regions of nucleotide sequences that are common to several alleles of the same and/or different loci. The DNA sequencing of a number of HLA alleles of various loci has also demonstrated that the majority of the variation is located in the α1 and α2 domain of the class I molecules and in the α1 and β1 domain of the class II molecules. These are called hyper variable regions.

A number of techniques have been developed to characterize these polymorphisms. Most of the described techniques make use of the polymerase chain reaction (PCR) to amplify the specific genes or region to be analysed. These techniques include PCR-SSP (PCR-sequence-specific priming), PCR-SSOP (PCR-sequence-specific oligonucleotide probing) and DNA sequencing-based typing (SBT).

The MHC Haplotype Consortium was originally formed with the aim of generating fully informative DNA polymorphisms and haplotype maps of the whole major histocompatibility complex (MHC) region (Stewart et al., 2004; Horton et al., 2004).

In the recent years, sequencing of the human genome and fine mapping of the MHC region identified variation in the form of single nucleotide polymorphisms (SNPs) which can be used for genotyping purposes. SNPs are bi-allelic markers and have a low rate of recurrent mutation which makes them relatively easy to genotype. SNPs are found in linkage disequilibrium with each other and background variation within the region they belong to. These types of SNPs are known as tagSNPs and tend to appear on haplotypes inherited together as a block on the same strand of a chromosome. Comparison of the haplotype sequences resulted in the identification of more than 44,000 variations. Subsequently, this information contributed towards the construction of a high-resolution LD map and a first generation of 7,500 HLA tag SNPs (Miretti et al., 2005; deBakker et al., 2006a and b).

Knowledge of these SNPs and their linkage-disequilibrium (LD) patterns enabled genome-wide association studies, which have successfully identified hundreds of novel genomic loci that influence human diseases (The International HapMap Consortium, 2003, 2004, 2005, 2007 and 2007).

More recent publication provided information of eight HLA homozygous haplotypes selected on the basis that they represented common haplotypes in the Northern European population (Horton et al., 2008; Bashal et al., 2008).

Previously described methods for the identification and definition of HLA alleles include PCR-SSP, PCR-rSSOP and SBT. Each of these methods has its own limitations but in general none of them are able to detect the HLA haplotypes. In order to detect HLA haplotypes, large number of HLA typed samples (for all HLA loci) and preferably of families, are required in order to apply the statistical methods that will give the linkage disequilibrium factor.

In addition, HLA alleles are extremely polymorphic and choosing a unique mutation to discriminate positive and negative samples for each of all HLA alleles is not possible.

The present invention seeks to address some of the problems in the currently available methods.

According to the present invention there is provided a method of genotyping a subject, the method comprising the steps of obtaining a sample from the subject, isolating the nucleic acid from the sample and identifying a selection of tag single nucleotide polymorphisms (SNPs) in a selection of human leukocyte antigen (HLA) haplotypes

Preferably, the subject is a human.

The HLA haplotypes and/or tagSNPs, may occur in chromosome 6 of a human subject. Preferably, the selection of HLA haplotypes are HLA-A*01-B*08-DRB1*03, HLA-A*02-B*44-DRB1*04 and HLA-A*03-B*07-DRB1*15.

Preferably, the selection of tagSNPs are Rs2734986, Rs2187668 for HLA-A*01-B*08-DRB1*0; Rs2844821, Rs2596477, Rs660895 for HLA-A*02-B*44-DRB1*04; and Rs3094170, Rs3130933 and Rs3129860 for HLA-A*03-B*07-DRB1*15.

These tagSNPs were selected because of their strong linkage disequilibrium with the common HLA haplotypes HLA-A*01-B*08-DRB1*03, HLA-A*02-B*44-DRB1*04 and HLA-A*03-B*07-DRB1*15. These three extended ancestral haplotypes have a combined frequency of over 10% in the Northern European Caucasoid population.

According to another aspect of the invention there is provided nucleotide sequences defined in SEQ ID NOs. 1 to 34.

According to a further aspect there is provided the use of the sequences defined in SEQ ID NO. 1 to 34 for the identification of tagSNPs in a sample.

For a sample, the region encompassing each of the tagSNPs were amplified using polymerase chain reaction methods and using a selection of nucleotide primers provided below corresponding to SEQ ID NOs. 1 to 16.

| | HLA | FORWARD | REVERSE | Size |
|---|---|---|---|---|
| tagSNP | allele | 5′-3′ | 5′-3′ | (bp) |
| rs2734986 | HLA- | TGCCTTTCTAGGGAGCAACCACT | TGGCTCCGCTTGGACCTTTG | 387 |
| | A*01 | | | |
| rs2187668 | HLA- | GTGAGGTGACACATATGAGGCAG | GGCTGAATGCCTTCAACAATCATTT | 75 |
| | DRB1*03 | | | |
| rs660895 | HLA- | TGCACAGAGTGAAGCCACCCA | ACTGCCTGCGGGTACTGCCT | 254 |
| | DRB1*04 | | | |
| rs2844821 | HLA- | TAACTCCTACTTGGCCAGACCT | TGCTCAGATGCCCACAGCACT | 480 |
| | A*02 | | | |
| rs2596477 | HLA-B*44 | ATAACTGCTTACAAGTGTGGCC | GTAGTAAGTCACTTAGATGAACATGA | 330 |
| rs3094170 | HLA- | AGAGCATTTGCCGAGGCCGA | GTCCAGCTCCTGGGTCCTCCC | 196 |
| | A*03 | | | |
| rs3129860 | HLA- | TCTCATCTTCACACTCCTTGTCTTCCT | ACGACAGTCATTTCTGCCACCTTT | 292 |
| | DRB1*15 | | | |
| rs3130933 | HLA- | GGGTGGGGGCAGGGGAGTTT | GGTGCAATCCCAGCTCTCCC | 271 |
| | B*07 | | | |

The major and minor alleles of each tagSNP were then identified in a sample using polymerase chain reaction methods and using a selection of allele specific nucleotide primers provided below designed for the use with MicroPlex TAG microspheres. Accordingly there is provided the sequences below corresponding to SEQ ID NOs. 17 to 32 and their uses thereof.

| | | ASPE | xTAG Bead |
|---|---|---|---|
| SNP ID | Nucleotide | Primer | IDs |
| RS2187668 | A | ACATATGAGGCAGCTGAGAGTAAA | LUA-006 |
| | G | CACATATGAGGCAGCTGAGAGTAAG | LUA-090 |
| RS2734986 | A | AGCACCAAAGCACCATTTCTTTA | LUA-019 |
| | G | TAAGCACCAAAGCACCATTTCTTTG | LUA-051 |
| RS660895 | G | CCAACAAAAACAAGACTTGTATG | LUA-030 |
| | A | CCAACAAAAACAAGACTTGTATA | LUA-033 |
| RS2844821 | G | CTTAGCAGTGCATCAGTGTCAATTG | LUA-068 |
| | A | CTTAGCAGTGCATCAGTGTCAATTA | LUA-012 |
| RS2596477 | A | TTCCTATTTTTTCCATATTCTTGACAA | LUA-008 |
| | G | TTCCTATTTTTTCCATATTCTTGACAG | LUA-088 |
| RS3094170 | G | CACCCCAAGGCTCTCTCCCATTAGG | LUA-028 |
| | A | CACCCCAAGGCTCTCTCCCATTAGA | LUA-077 |
| RS3129860 | A | GCTAACCATGTACCTTAAATAAACCA | LUA-076 |
| | G | GCTAACCATGTACCTTAAATAAACCG | LUA-018 |
| RS3130933 | A | GTTTGTCCTTCAGTTACTGAGGTA | LUA-059 |
| | G | TTGTCCTTCAGTTACTGAGGTG | LUA-029 |

The major and minor alleles of each tagSNP can also be identified in a sample using polymerase chain reaction methods and using a selection of allele specific nucleotide primers provided below for the use of Luminex MagPlex TAG microspheres.

| | | | MagPlex- |
|---|---|---|---|
| | | | TAG |
| | | ASPE | microsphere |
| SNP ID | Nucleotide | Primer Sequence | ID |
| RS2187668 | A | ACATATGAGGCAGCTGAGAGTAAA | MTAG-A026 |
| | G | ATATGAGGCAGCTGAGAGTAAG | MTAG-A019 |
| RS2734986 | A | AGCACCAAAGCACCATTTCTTTA | MTAG-A013 |
| | G | TAAGCACCAAAGCACCATTTCTTTG | MTAG-A012 |
| RS660895 | G | CCAACAAAAACAAGACTTGTATG | MTAG-A030 |
| | A | CCAACAAAAACAAGACTTGTATA | MTAG-A022 |
| RS2844821 | G | TTAGCAGTGCATCAGTGTCAATTG | MTAG-A014 |
| | A | CTTAGCAGTGCATCAGTGTCAATTA | MTAG-A015 |
| RS2596477 | A | TTCCTATTTTTTCCATATTCTTGACAA | MTAG-A029 |
| | G | TTCCTATTTTTTCCATATTCTTGACAG | MTAG-A027 |
| RS3094170 | G | CACCCCAAGGCTCTCTCCCATTAGG | MTAG-A033 |
| | A | CACCCCAAGGCTCTCTCCCATTAGA | MTAG-A025 |
| RS3129860 | A | GCTAACCATGTACCTTAAATAAACCA | MTAG-A020 |
| | G | GCTAACCATGTACCTTAAATAAACCG | MTAG-A021 |
| RS3130933 | A | GTTTGTCCTTCAGTTACTGAGGTA | MTAG-A018 |
| | G | TTGTCCTTCAGTTACTGAGGTG | MTAG-A028 |

The identification method may use the ASPE-Luminex assay which may be optimised for genotyping the selected tagSNPs

The optimisation steps may include the optimisation of annealing and extension temperature, dNTPs concentration, MgCl₂ concentration and addition of PCR additives. It would be within the remit of a person skilled in the art to optimise these conditions as needed.

The method may be validated with a panel of homozygous and heterozygous DNA samples for each haplotype as necessary.

The terminology used in the specification would be within the understanding of a person skilled in the art and some terms are described for clarity only.

HLA Haplotype: set of HLA alleles segregating in strong linkage disequilibrium at a frequency higher that that expected by random segregation. This normally occurs with alleles of closely located genes.

TagSNP: SNP defining an specific allele or in close linkage with a particularly allele

ASPE assay: Allele specific primer extension (ASPE) assay relies on the sequence-specific primer extension of two allele-specific capture oligonucleotide primers that differ at their 3′-end nucleotide defining the allele

Multiplex Assay format; where primers and reagents were tested simultaneously in a single tube reaction.

Based on the strong linkage disequilibrium within the MHC region, the inventors have shown that a small panel of unique tagSNPs (n=8) can be used to identify 3 of the most common Northern European (NE) Caucasoid HLA haplotypes. The inventors have also combined the 8 tagSNP in a multiplex assay for the simultaneous detection of these SNPs using the Luminex platform.

Thus, the inventors have further developed a rapid method that allows the simultaneous detection of the 3 most common HLA haplotypes present in the Northern European Caucasoid population using a set of 8 tag SNPs.

Three common Caucasian haplotypes, HLA-A*01-B*08-DRB1*03, HLA-A*02-B*44-DRB 1*04 and HLA-A*03-B*07-DRB1*15, were selected and the tagSNPs were genotyped. In particular, the tagSNPs selected were in strong linkage disequilibrium with the common HLA haplotypes. These three extended ancestral haplotypes have a frequency of over 10% in the North European Caucasoid population.

The present inventors surprisingly found that the present panel of eight SNPs identified three of the most frequent HLA haplotypes in the Northern European Caucasian population. A multiplex ASPE-Luminex assay was optimised to genotype these eight SNPs in samples positive and negative for each of the three HLA haplotypes. Rs2187668 and rs2734986 were selected to identify HLA-A*01-B*08-DRB1*03. In both controls and a blind sample cohort, no discrepancies were found between the tagSNP ASPE-Luminex genotyping assay and previous genotyping method. A tagSNP for HLA-B*08 allele was not needed for tagging the HLA-A*01-B*08-DRB1*03 haplotype which demonstrates the strong linkage disequilibrium between the HLA alleles. When HWE was calculated for each of the two tagSNPs, the p value for rs2734986 was found to be less than 0.05. This result can be explained by the strong LD between rs2734986 and the HLA-A*01 allele and therefore the occurrence of this SNP in the population is not independent of the haplotype in which it appeared first time.

Rs2844821, rs2596477 and rs660895 which tag HLA-A*02, HLA-B*44 and HLA-DRB1*04 were selected to identify the HLA-A*02-B*44-DRB1*04 haplotype. The selected tagSNPs identified HLA-A*02-B*44-DRB1*04 in all homozygous controls tested. In the heterozygous controls, rs660895 that tags the HLA-DRB1*04 allele also tags the HLA-DRB1*08 allele. However, based on the strong LD between the HLA-A*02, HLA-B*44 and HLA-DRB1*04 alleles, there was a preference of these 3 alleles to be inherited together as a haplotype. The occurrence of the HLA-B*08 together with HLA-A*02 and HLA-B*44 was found to be less frequent than that of the HLA-DRB1*04 allele.

The ability to select match unrelated individuals, not only for the HLA alleles but also for the complete HLA haplotype using specific SNPs could have benefits in the clinical outcome of haematopoietic stem cell transplantation between individuals with identical extended haplotypes. In the case of donors of blood products such as platelets, red cells or white cells (granulocytes, T cells), this method could be used to select homozygous donors expressing common HLA haplotypes which are required and very useful for the provision of HLA matched blood and blood products.

This method may also be used to identify disease and drug susceptible HLA alleles and haplotypes in patients and normal individuals. It could also be used to trace the evolution and migration of HLA haplotypes in certain human populations.

TagSNPs found in linkage disequilibrium with HLA loci can also be used to identify and genotype these HLA genes. SNP based genotyping offers an attractive alternative to conventional HLA typing when 100% accuracy in allele typing is not required.

The strong haplotype structure observed across the MHC region can be used as an alternative approach to predicting HLA types. For two chromosomes that have extensive SNP identity across an HLA locus usually share the same HLA alleles. Therefore, informative tagSNPs can give information about extended ancestral HLA haplotypes within an ethnic group.

The low rate of mutation makes SNPs stable markers and therefore preferred tools for genotyping purposes

The MHC region contains a number of extended or “ancestral” haplotypes with linked HLA alleles (Delki-Esposi et al., 1992, Carrington M et al., 1994, Dawkins R et al., 1999, Dorak M T et al., 2006 and Bashal E E et al., 2008). Conserved extended haplotypes were extremely common and many of them can be detected with panels of specific tagSNPs. This has been demonstrated previously for haplotype HLA-A*01-B*08-DRB1*03 (Smith W P et al., 2006; Aly T A et al., 2008). Aly T A et al. (2008) used a panel of 12 tagSNPs and detected the haplotype with a specificity of 95%.

A further aspect of the invention is the optimisation of a multiplex ASPE-Luminex assay for SNP genotyping using unique primers.

The method of genotyping may additionally comprise one or more of the steps of selection of tagSNPs, designing generic primers to capture the region where tagSNPs are located, optimising the primers in both single and multiplex format, designing allele specific primer extension (ASPE) primers for each tagSNP, selecting the xTAG beads to bind and detect each tagSNP, optimising and validating the ASPE primers in both a singleplex and all together in a multiplex assay and further validating against a panel of homozygous and heterozygous DNA samples for each of the three common haplotypes in order to determine MFI and allelic ratio cut-offs for each individual SNP. The method may further comprise the step of validating against DNA samples from an HLA typed panel of cells by genotyping and determining allele frequencies and p values for Hardy-Weinberg equilibrium (HWE) in samples of unrelated North European Caucasoid subjects.

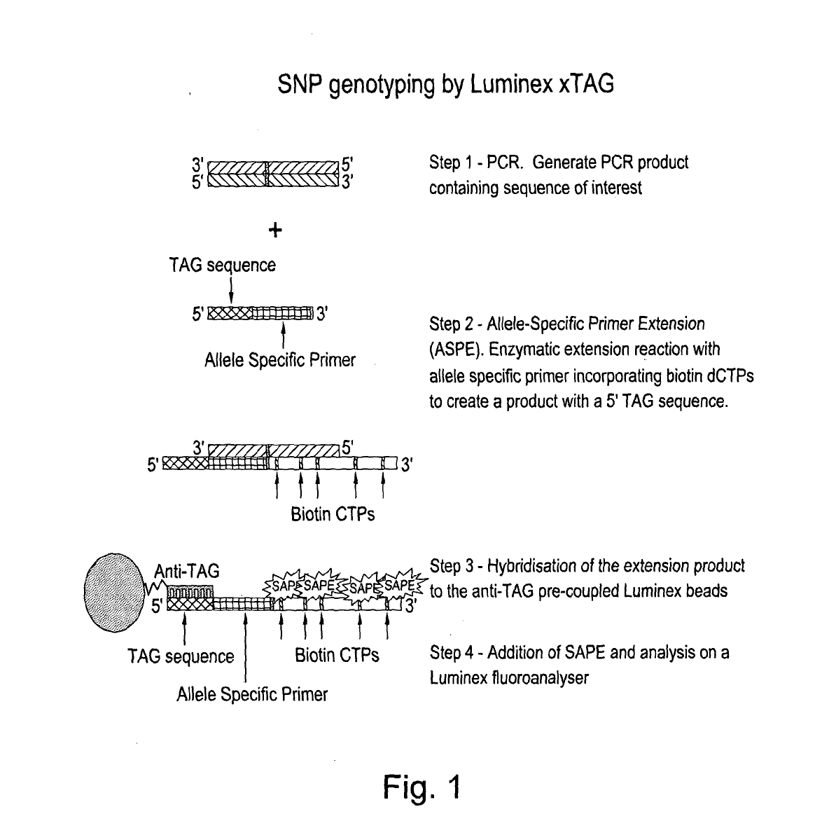

FIG. 1 shows a schematic of the ASPE-Luminex assay. The region encompassing each of the tagSNPs is captured by PCR and the amplified product is used as target DNA for the ASPE reaction. The ASPE reaction relies on the sequence-specific primer extension of two allele-specific capture oligonucleotide primers that differ at their 3′-end nucleotide defining the allele. During the ASPE reaction, ASPE primers will anneal to the target DNA and extension of the primers will occur only if the 3′ end is complimentary to the target DNA. Extended DNA products are incorporated with biotin-labelled CTP and are captured on unique xTAG beads. Upon hybridisation with Streptavidin-conjugated Phycoerythrin (SAPE), the Luminex fluoroanalyser records the Median Fluorescence Intensity signal (MFI) and identifies the beads analysed. Therefore for each HLA tagSNP, 2 different ASPE primers were designed with one having the major allele nucleotide at the 3′ end and the other having the minor allele nucleotide present. As a result, homozygous, heterozygous and negative samples for each HLA haplotype can be simultaneously detected in one single reaction, making the process faster and cheaper than those currently available.

It has been reported that the ASPE method has the disadvantage of poor amplification efficiency. Low amplification product can influence the efficiency of the ASPE reaction and assigning allele genotypes. We therefore chose to combine the ASPE with Luminex xTAG technology for developing the HLA haplotype tagSNP assay since the method is simple and easily optimised. The ASPE method is designed for SNP genotyping enabling extension of product only when the allele of interest is present and the specific nucleotide is complementary to the 3′end of the discriminating ASPE primer. The availability of Luminex xTAG beads with specifically designed and validated TAG and anti-TAG sequences that will allow hybridization to occur at 37° C. eliminates long optimisation procedures.

The present inventors have optimised the use of these SNPs in a multiple ASPE-Luminex assay for the simultaneous detection of 8 HLA tagSNPs (and 3 HLA haplotypes) in one single reaction/tube.

Accordingly, the present invention can be modified for high throughput screening of samples for haplotyping potential donors.

According to another aspect of the invention there is provided a kit for HLA haplotyping a human subject comprising both tagSNP primers and ASPE primers. PCR reagents can be additionally provided. For an ASPE-Luminex assay, the kit can further comprise ASPE reagents, Luminex xTAG beads, hybridization buffer and SAPE.

The sequences in SEQ ID NOs 1 to 32 can be provided in the kit. SEQ ID NOs 1 to 16 are the tag SNP primers. SEQ ID NOs 17 to 32 are the ASPE primers.

The invention will now be described by way of illustration only in the following examples in which:

FIG. 1 is a schematic showing the SNP genotyping by Luminex xTAG

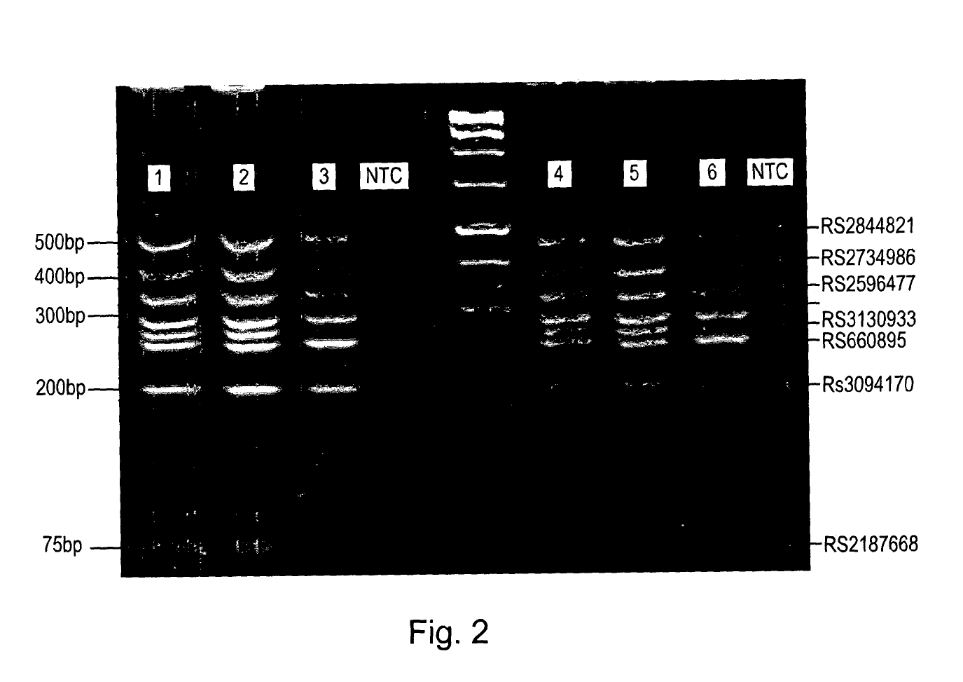

FIG. 2 Shows an agarose gel electrophoresis with the PCR products following optimisation of dNTP concentration for the tagSNPs multiplex PCR reaction. Samples 1 to 5 were amplified with 300 μM of each dNTP and samples 6 to 10 were amplified using 400 μM of each dNTP. Red arrows indicate PCR amplicons corresponding to each tagSNP; NTC represents no template control; 1 KB DNA ladder in lanes 1 and 7.

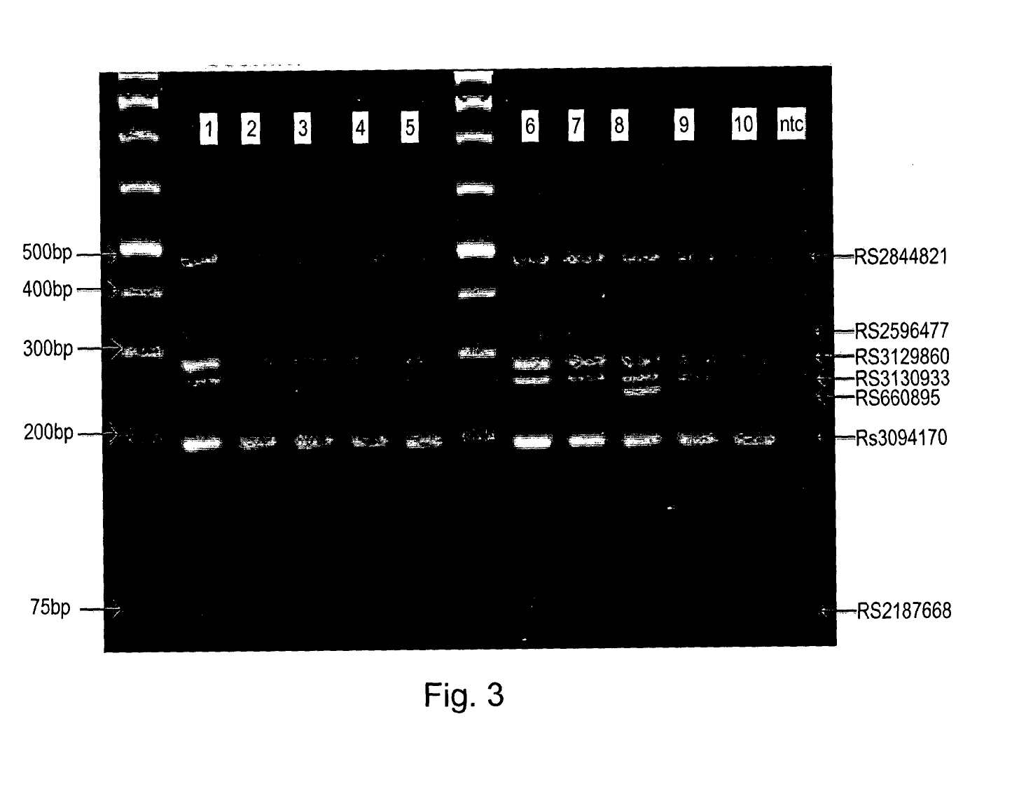

FIG. 3 Shows an agarose gel electrophoresis with the PCR products following extension temperature optimisation for the tagSNPs multiplex PCR reaction. Samples 1 to 3 were amplified using an extension temperature of 67° C.; samples 4 to 6 were amplified using an extension temperature of 69° C. Red arrows indicate PCR products corresponding to each of the 8 tagSNPs; 1 Kb ladder is in lane 5.

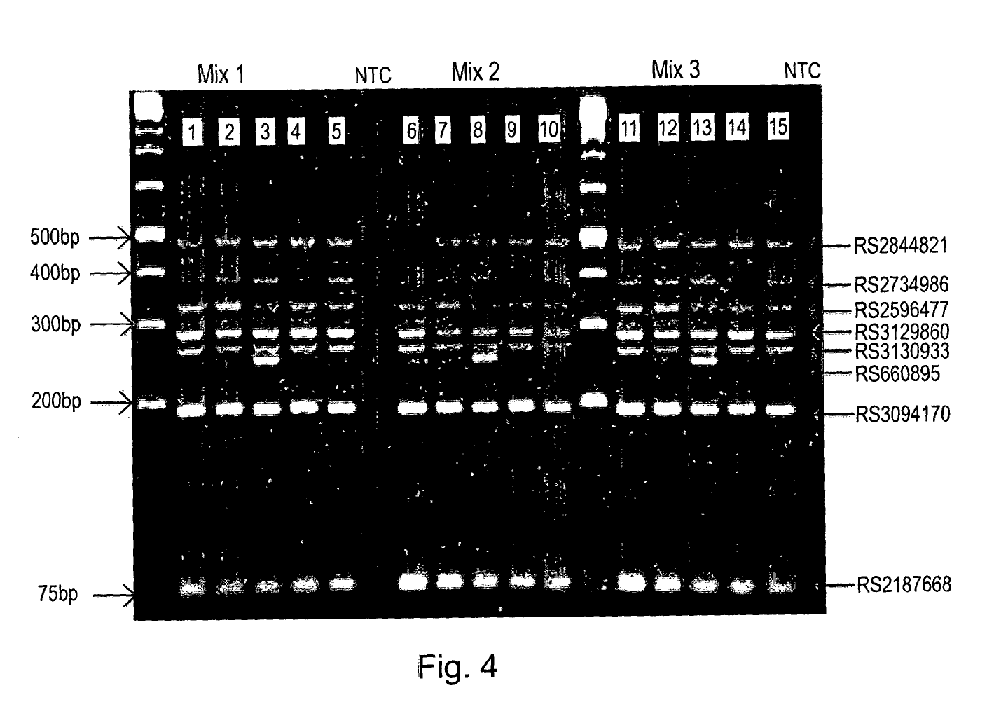

FIG. 4 Shows an agarose gel electrophoresis with the PCR products following optimisation of BSA concentration for the tagSNP multiplex PCR reaction

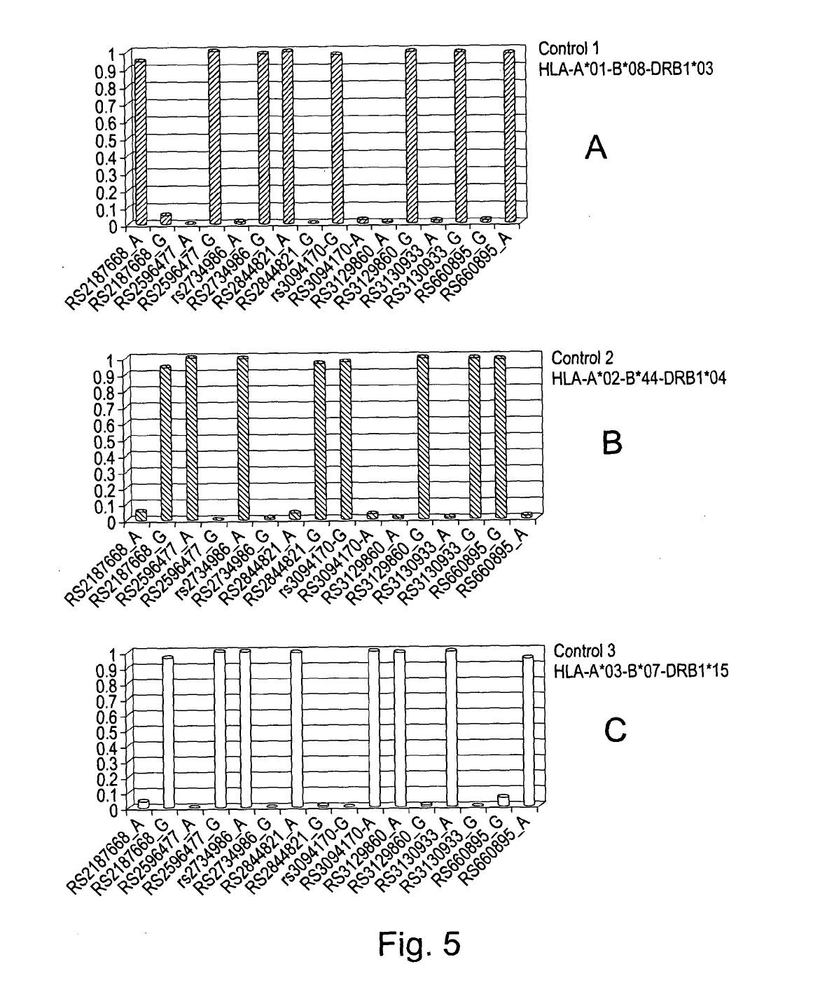

FIG. 5A-5C are graphs of the tagSNPs multiplex ASPE-Luminex assay for the three homozygous haplotype controls A) Multiplex ASPE-Luminex results for HLA-A*01-B*08-DRB1*03 Control 1; B) Multiplex ASPE-Luminex results for HLA-A*02-B*44-DRB1*04 Control 2; C) Multiplex ASPE-Luminex results for HLA-A*03-B*07-DRB1*15 Control 3. Allelic ratios values were represented on the vertical axis. TagSNPs alleles are shown on the horizontal axis.

### EXAMPLES

### Example 1

### tagSNPs Selection

STATA program [STATA software (http://www.stata.com/) was used to select tagSNPs for the HLA alleles based on their LD coefficient r². All tagSNPs were located on chromosome 6 and the corresponding chromosomal positions are shown in Table 1. The minor alleles of the SNPs, tag the corresponding HLA allele and haplotype. LD r² for all SNP were above 0.9 and the values are listed in Table 1. The specificity and location of the tagSNPs are shown in Table 1.

TABLE 1
Chromosomal location, allele frequencies and linkage disequilibrium
coefficient for selected 8 tagSNPs

| | CHROMO- | | MINOR | | |
|---|---|---|---|---|---|
| | SOME | HLA | AL- | MAJOR | LD/ |
| SNP ID | POSITION | ALLELE | LELE | ALLELE | R² |
| Rs2734986 | 6: 29,926,547 | HLA-A*01 | G: 0.17 | A: 0.63 | 0.94 |
| Rs2187668 | 6: 32,713,862 | HLA- | A: 0.1 | G: 0.9 | 0.98 |
| | | DRB1*03 | | | |
| Rs2844821 | 6: 29,946,621 | HLA-A*02 | G: 0.36 | A: 0.64 | 0.9 |
| Rs2596477 | 6: 31,435,702 | HLA-B*44 | A: 0.2 | G: 0.8 | 0.97 |
| Rs660895 | 6: 32,685,358 | HLA- | G: 0.22 | A: 0.78 | 0.9 |
| | | DRB1*04 | | | |
| Rs3094170 | 6: 29,937,244 | HLA-A*03 | A: 0.01 | G: 0.99 | 0.9 |
| Rs3130933 | 6: 31,240,064 | HLA-B*07 | A | G (0.99) | 0.94 |
| | | | (0.01) | | |
| Rs3129860 | 6: 32,509,057 | HLA- | A | G (0.99) | 0.9 |
| | | DRB1*15 | (0.01) | | |

TagSNPs were selected using SNP data generated from Affymetrix (2K) (Santa Clara, Calif.) and Illumina (500K) (San Diego, Calif.) gene chips following the typing of a British Birth Control Cohort. This is a bank of DNA samples from approximately 2,000 individuals of NE Caucasoid ancestry funded by the Wellcome Trusts and the MRC). HLA haplotypes were identified using Haplo.Stats package in the R statistical software (http://www.r-project.org/).

The tagging specificity of the SNPs for the haplotype was estimated using PHASE software (Stephens M et al., 2001). In this panel, the control British Birth cohort, HLA-A*0101-B*0801-DRB1*0301 has a frequency of 8.345%. rs2734986 and rs2187668 which tags HLA-A*01 and HLA-DRB1*01 can identify this haplotype with a specificity of 96%, which indicates that just under 4% of this haplotype will be missed. Thus, due to the strong linkage disequilibrium between the HLA alleles only 2 SNPs were needed to identify HLA-A*0101-B*0801-DRB1*0301. Therefore, a SNP which tags HLA-B*08 allele will not increase the prediction percentage for this haplotype.

The HLA-A*0201-B*4402-DRB1*0401 haplotype has a frequency of 4.89% in the British Birth Control cohort and the tagSNPs rs2844821, rs2596477 and rs660895 identify this haplotype with a specificity of 98.67%, which indicates that just under 2% of all HLA-A*0201-B*4402-DRB1*0401 haplotypes are not captured with these 3 SNPs.

HLA-A*0301-B*0701-DRB1*1501 has a frequency of 0.03578% in the British 1958 Birth cohort. rs3094170, rs3130933 and rs3129860 identify this haplotype with a specificity of 100% which indicates that all HLA-A*0301-B*0701-DRB1*1501 samples will be captured.

The tagging specificity of the SNPs for the haplotype was estimated using PHASE software (Stephens M et al., 2001).

### Example 2

### Design of tagSNP Generic Primers to Capture the Region where SNPs are Located

TagSNP generic primers were designed using DNASIS Smartnote online bioinformatics tool (http://smartnote.miraibio.com/index.htm) and the primer sequences were shown below in Table 2.

TABLE 2
Primers for each of the eight tagSNPs in Table 1

| | HLA | FORWARD | REVERSE | Size |
|---|---|---|---|---|
| tagSNP | allele | 5′-3′ | 5′-3′ | (bp) |
| rs2734986 | HLA- | TGCCTTTCTAGGGAGCAACCACT | TGGCTCCGCTTGGACCTTTG | 387 |
| | A*01 | | | |
| rs2187668 | HLA- | GTGAGGTGACACATATGAGGCAG | GGCTGAATGCCTTCAACAATCATTT | 75 |
| | DRB1*03 | | | |
| rs660895 | HLA- | TGCACAGAGTGAAGCCACCCA | ACTGCCTGCGGGTACTGCCT | 254 |
| | DRB1*04 | | | |
| rs2844821 | HLA- | TAACTCCTACTTGGCCAGACCT | TGCTCAGATGCCCACAGCACT | 480 |
| | A*02 | | | |
| rs2596477 | HLA-B*44 | ATAACTGCTTACAAGTGTGGCC | GTAGTAAGTCACTTAGATGAACATGA | 330 |
| rs3094170 | HLA- | AGAGCATTTGCCGAGGCCGA | GTCCAGCTCCTGGGTCCTCCC | 196 |
| | A*03 | | | |
| rs3129860 | HLA- | TCTCATCTTCACACTCCTTGTCTTCCT | ACGACAGTCATTTCTGCCACCTTT | 292 |
| | DRB1*15 | | | |
| rs3130933 | HLA- | GGGTGGGGGCAGGGGAGTTT | GGTGCAATCCCAGCTCTCCC | 271 |
| | B*07 | | | |

Generic PCRs were optimised for each individual tagSNP. Standard materials and methods were used for the PCR method. Optimisation of the PCR conditions, as necessary, would be within the skills of a person skilled in the art.

Annealing temperature gradient PCRs between 55° C. and 61° C. were performed to test each individual tagSNP primer pair. Amplicons of the expected sizes were amplified for all the tagSNP regions with the four annealing temperatures tested. For rs2734986 and rs2596477, faint PCR products were observed at 55° C. At annealing temperatures of 55° C. and 57° C., non-specific amplification is observed for rs3130933. Therefore, an annealing temperature of 59° C. was chosen for further optimisation and validation.

### Example 3

### Multiplex PCR Reaction Optimisation

The primers listed in Table 2 were combined into a multiplex PCR reaction. Each primer pairs were designed to amplify different amplicon sizes to enable further use in a multiplex assay. Standard materials and methods were used for the PCR method. Optimisation of the PCR conditions, as necessary, would be within the skills of a person skilled in the art.

Initially all primers in the reaction mixtures were used at a concentration of 200 nM. In a series of initial experiments, it was observed that at 200 nM, some of the primers amplified better than others so the concentration of primers added to the PCR was increased or decreased accordingly.

When different primer concentrations were used, amplification efficiency for some SNP loci were improved but not for all of them and therefore further optimisation was necessary. The parameters optimised include the extension time and temperature, the concentration of dNTPs, BSA and MgCl₂.

A concentration of 1.5 mM MgCl₂ was used initially for the multiplex assay, however different amplification efficiencies were observed. Therefore, amplification with 3 mM and 4 mM MgCl₂ were tested (data not shown). PCR amplification appeared to be more efficient at 4 mM and therefore was used for further validation. However, no PCR amplicon was observed for SNP rs2734986. The next step of the multiplex PCR optimisation was to vary the dNTP concentration.

Multiplex PCR reactions were set up with 300 μM (sample 1-5) and 400 μM (samples 6-10) of each dNTP. FIG. 2 shows that PCR amplification was observed for all loci except for the amplicon corresponding to rs2734986. At 300 μM dNTP concentration, the 330 bp amplicon corresponding to rs660895 appears to amplify less efficiently than the other 6 amplified products. Overall, PCR amplification appears to be more efficient with 400 μM dNTP concentration and was subsequently used for further validation. The PCR extension time was also increased from 30 seconds to 1 minute.

However, with 400 μM of each dNTP, 4 mM MgCl₂ and an extension time of 1 minute, no amplification was observed for rs2734986. Extension time was increased from 1 minute to 2 minutes and extension temperature was decreased to 69° C. and 67° C. Following these changes PCR amplification was successful for all SNP loci including rs2734986.

FIG. 3 shows the results for multiplex PCR reaction with an extension temperature of 67° C. and 69° C. Samples 1 to 3 were amplified at 67° C. while samples 4 to 7 were amplified using 69° C. for the extension temperature. Amplification is observed for all 8 tagSNP loci at both extension temperatures tested. At 67° C., amplification appears to be more efficient and therefore was subsequently used for further validation and optimisation of the assay.

To further improve the amplification of the multiplex PCR reaction, BSA concentration was also optimised and results are shown in FIG. 4. Samples 1-5 were amplified with the addition of 0.4 μg/μl BSA, samples 6-10 were amplified with 0.6 μg/μl BSA and for samples 11-15, a BSA concentration of 0.8 μg/μl was added. FIG. 4 shows that there is amplification with all 3 concentrations tested but the most efficient amplification for all 8 SNP loci is observed with 0.4 μg/μl BSA.

Therefore, the optimal PCR reaction conditions for the multiplex assay were: 4 mM MgCl₂, 400 μM dNTPs, 0.4 μg/μl BSA, 59° C. annealing temperature and 67° C. extension step for 2 minutes followed by final extension for 5 minute at 67° C. Using these parameters similar amplification efficiencies were achieved for all 8 SNPs amplicon products. This step is very important because poor amplification efficiency and low amplification product can influence the efficiency of the ASPE reaction and with assigning allele genotypes.

Multiplex PCR amplification was performed in a final volume of 50 μl consisting of 1× Gold PCR buffer II (Applied Biosystems, Foster City, UK), 4 mM MgCl₂ (Applied Biosystems, Foster City, Calif.), 400 μM each dNTP (Invitrogen, UK), 100 nM rs2844821, 200 nM rs2187668 primers, 600 nM rs2734986 primers, 300 nM rs660895 primers, 240 nM rs2596477 primers, 60 nM rs3094170 primers, 150 nM rs3130933 primers, 100 nM rs3129860 primers, 0.4 μg/μl BSA, 2.5 U of AmpliTaq Gold polymerase (Applied Biosystems, Foster City, UK) and 150 ng of gDNA. PCR cycling parameters are shown in Table 3.

TABLE 3
PCR Cycling parameters for tagSNPs multiplex reaction

| Steps | Temperature (° C.) | Time (seconds) | | Cycles |
|---|---|---|---|---|
| Initial denaturation | 95 | 5 | min | 1 |
| Denaturation | 95 | 30 | s | 35 |
| Annealing | 59 | 45 | s | |
| Extension | 67 | 2 | min | |
| Final extension | 67 | 5 | min | 1 |

### Example 4

### Multiplex ASPE-Luminex Assay Optimisation and Validation

ASPE multiplex reactions were carried out by hybridising all 8 SNP amplicons with all 8 ASPE primer pairs, each with a 5′TAG sequence corresponding to an xTAG bead. ASPE primers used to detect the alleles of each tagSNP are shown in Table 4.

ASPE products were subsequently hybridised with all selected beads for Luminex detection. MFI signals are generated upon hybridisation of the 5′TAG sequence of the ASPE primers to the anti-TAG sequence attached to the beads and incubation with SAPE. The xTAG microsphere beads and corresponding TAG and anti-TAG nucleotide sequences may be as shown in Table 5

TABLE 4
Nucleotide sequences of ASPE primer for tagSNPs

| | | ASPE | xTAG Bead |
|---|---|---|---|
| SNP ID | Nucleotide | Primer | IDs |
| RS2187668 | A | ACATATGAGGCAGCTGAGAGTAAA | LUA-006 |
| | G | CACATATGAGGCAGCTGAGAGTAAG | LUA-090 |
| RS2734986 | A | AGCACCAAAGCACCATTTCTTTA | LUA-019 |
| | G | TAAGCACCAAAGCACCATTTCTTTG | LUA-051 |
| RS660895 | G | CCAACAAAAACAAGACTTGTATG | LUA-030 |
| | A | CCAACAAAAACAAGACTTGTATA | LUA-303 |
| RS2844821 | G | CTTAGCAGTGCATCAGTGTCAATTG | LUA-068 |
| | A | CTTAGCAGTGCATCAGTGTCAATTA | LUA-012 |
| RS2596477 | A | TTCCTATTTTTTCCATATTCTTGACAA | LUA-008 |
| | G | TTCCTATTTTTTCCATATTCTTGACAG | LUA-088 |
| RS3094170 | G | CACCCCAAGGCTCTCTCCCATTAGG | LUA-028 |
| | A | CACCCCAAGGCTCTCTCCCATTAGA | LUA-077 |
| RS3129860 | A | GCTAACCATGTACCTTAAATAAACCA | LUA-076 |
| | G | GCTAACCATGTACCTTAAATAAACCG | LUA-018 |
| RS3130933 | A | GTTTGTCCTTCAGTTACTGAGGTA | LUA-059 |
| | G | TTGTCCTTCAGTTACTGAGGTG | LUA-029 |

TABLE 5
TAG and anti-TAG sequences of selected Luminex xTAG beads

| | TAG nucleotide sequences | Anti-TAG nucleotide |
|---|---|---|
| Microsphere | at the 5′ end | sequences covalently |
| beads | of the ASPE primer | attached to the xTAG bead |
| LUA-006 | TCAACAATCTTTTACAATCAAATC | GATTTGATTGTAAAAGATTGTTGA |
| LUA-090 | CTAAATACTTCACAATTCATCTAA | TTAGATGAATTGTGAAGTATTTAG |
| LUA-051 | TCATTTCAATCAATCATCAACAAT | ATTGTTGATGATTGATTGAAATGA |
| LUA-019 | TCAATCAATTACTTACTCAAATAC | GTATTTGAGTAAGTAATTGATTGA |
| LUA-030 | TTACCTTTATACCTTTCTTTTTAC | GTAAAAAGAAAGGTATAAAGGTAA |
| LUA-033 | TCAATTACTTCACTTTAATCCTTT | AAAGGATTAAAGTGAAGTAATTGA |
| LUA-012 | TACACTTTCTTTCTTTCTTTCTTT | AAAGAAAGAAAGAAAGAAAGTGTA |
| LUA-068 | TCATAATCTCAACAATCTTTCTTT | AAAGAAAGATTGTTGAGATTATGA |
| LUA-008 | AATCCTTTTACATTCATTACTTAC | GTAAGTAATGAATGTAAAAGGATT |
| LUA-088 | TTACTTCACTTTCTATTTACAATC | GATTGTAAATAGAAAGTGAAGTAA |
| LUA-028 | CTACAAACAAACAAACATTATCAA | TTGATAATGTTTGTTTGTTTGTAG |
| LUA-029 | AATCTTACTACAAATCCTTTCTTT | AAAGAAAGGATTTGTAGTAAGATT |
| LUA-059 | TCATCAATCAATCTTTTTCACTTT | AAAGTGAAAAAGATTGATTGATGA |
| LUA-018 | TCAAAATCTCAAATACTCAAATCA | TGATTTGAGTATTTGAGATTTTGA |
| LUA-076 | AATCTAACAAACTCATCTAAATAC | GTATTTAGATGAGTTTGTTAGATT |
| LUA-077 | CAATTAACTACATACAATACATAC | GTATGTATTGTATGTAGTTAATTG |

MFI values generated from the multiplex ASPE-Luminex assay can then be used to determine allelic ratios and assign genotypes. For each allele of a given sample, the net MFI was obtained by subtracting the no-template control MFI values from the corresponding MFI values of the test sample. For each tagSNP, MFI values for at least one allele were required to be at least 200, 10 times greater than for no-template DNA for that particular allele. For tagSNPs meeting this criterion, the genotype was determined by the mutant allelic ratio whereby:

Mutant allelic ratio=(NET MFI)_{mutant allele}/(NET MFI)_{mutant allele}+(NET MFI)_{wild type allele}

For the initial validation, arbitrary cut-offs determined by the Luminex Corporation were used. The allelic ratio ranges were set to 0.00-0.25 for assigning homozygous wild-type calls, 0.25-0.75 for heterozygous calls and 0.75-1.00 for homozygous mutant calls.

Multiplex ASPE-Luminex reaction were optimised and validated with homozygous and heterozygous samples characteristic for all 3 common haplotypes. Optimisation of the PCR conditions, as necessary, would be within the skills of a person skilled in the art.

Initially all ASPE primers were used at the recommended concentration of 25 nM. After initial validation, ASPE primer concentration was increased to 50 nM for rs2844821 allele G, rs2734986 allele G and rs2596477 both alleles A and G. These 4 ASPE primers have shown lower hybridisation efficiencies and therefore lower MFI values compared with the MFI values for all the other tagSNPs (data not shown).

The multiplex ASPE reaction was subsequently carried out in a final volume of 20 μl and consisted of 1×ASPE buffer, 1.25 mM MgCl₂, 25 nM rs2187668, rs660895 rs3094170, rs3129860, rs3130933 ASPE primers, 50 nM rs2596477, rs2734986 allele G, rs2844821 allele G ASPE primers, 12.5 nM rs2734986 allele A, rs2844821 allele A ASPE primers, 5 μM each of dATP, dGTP, dTTP, and biotin-dCTP, 0.3 U Tsp DNA polymerase and 5 μl of PCR product treated with ExoSAP. ASPE cycling parameters used are shown in Table 6.

TABLE 6
PCR cycling parameters for the multiplex ASPE reaction

| Steps | Temperature (° C.) | Time | No. of Cycles |
|---|---|---|---|
| Initial denaturation | 96 | 1 min | 1 |
| Denaturation | 94 | 30 s | 30 |
| Annealing | 55 | 60 s | |
| Extension | 74 | 2 min | |

A panel of 59 samples homozygous at all three HLA loci for each of the 3 common Caucasian haplotypes were used to validate the multiplex ASPE reaction. 20 samples were homozygous positive for all 3 loci of the HLA-A*01-B*08-DRB1*03 haplotype, 20 samples were homozygous for the HLA-A*02-B*44-DRB1*04 haplotype and 19 samples were homozygous for all 3 HLA loci for the HLA-A*03-B*07-DRB1*03 haplotype. A panel of 71 heterozygous DNA controls were also tested. HLA genotypes for these homozygous and heterozygous control samples were assigned using resulting allelic ratios. The HLA genotypes assigned by the ASPE-Luminex assay were compared with previous genotypes determined by the H&I laboratory in Colindale. Genotypes were considered positive if the SNP allele determined by the Luminex assay corresponded with the nucleotide sequence generated by high resolution SBT typing. For SNPs rs2187668 and rs2844821, PCR-SSP was performed instead of SBT. No discrepancy was observed between the methods used.

Differences in hybridisation efficiency were observed between the two alleles of the SNPs for rs2734986, rs2844821 and rs3094170 in the heterozygous controls. Further optimisation was conducted to achieve similar hybridisation efficiencies for both alleles of these 3 SNPs. The optimisation consisted in increasing the amount of ASPE primer for the allele that expressed lower MFIs, increasing the xTAG microsphere beads hybridisation time, increasing the amount of biotin-labelled dCTP incorporated and SAPE concentration. In all cases no major differences were observed.

An example of a tagSNPs multiplex ASPE-Luminex reaction for three homozygous controls is presented in FIG. 5. FIG. 5.A shows the multiplex ASPE-Luminex results for an HLA-A*01-B*08-DRB1*03 positive control. Control 1 DNA expressed the minor alleles A and G for tagSNPs rs2187668 and rs2734986 respectively. All the remaining six tagSNPs expressed the major alleles and as a result control 1 is a homozygous HLA-A*01-B*08-DRB1*03 sample.

Control 2 DNA expressed the minor alleles for rs2844821 (G), rs2596477 (A) and rs660895(G) and the major alleles for the remaining five SNPs (FIG. 5.B). Therefore, control 2 is an HLA-A*02-B*44-DRB1*04 DNA sample.

Control 3 expresses the minor alleles for rs3094170 (A), rs3129860 (A), rs3130933 (A) and the major alleles for the remaining tagSNPs. For that reason, control 3 has the genotype HLA-A*03-B*07-DRB1*15 (FIG. 5.C) See Table 1 for definition of major and minor alleles for each tagSNP.

### Example 5

### Establishing MFI and Allelic Ratio Cut-Offs for the tagSNP Multiplex ASPE-Luminex assay

In the initials stages of assay development and validation, arbitrary Luminex cut-offs were used to assign genotypes for each individual tagSNPs in the multiplex reaction. Subsequently, the homozygous and heterozygous controls were used to determine MFI and allelic ratio values cut-offs specific for each tagSNPs of this assay (Table 7). The cut-offs were determined based on the average MFI and allelic ratio values obtained for each of the eight SNP allele in separate homozygous and heterozygous samples. The cut-off value represents the lower value at which a SNP genotype can be assigned. Any value above the established cut-off is considered positive and a genotype can be made based on it.

Based on the allelic ratios calculated in the heterozygous samples cohort, the cut-off was adjusted to 0.20 for SNPs rs2734986, rs2844821 and rs3094170. The MFI and allelic ratio cut-offs can then be applied to determined the HLA genotypes in samples as required.

TABLE 7
MFI and Allelic Ratio cut-offs values for the tagSNPs

| | Homozygous | | | | Heterozygous | | | |
|---|---|---|---|---|---|---|---|---|
| | controls | | | | controls | | | |
| SNP ID | MFI | | Allelic ratios | | MFI | | Allelic ratio | |
| | Allele A | Allele G | Allele A | Allele G | Allele A | Allele G | Allele A | Allele G |
| Rs2187668 | 1000 | 1000 | 0.90-1.0 | 0.90-1.0 | 1000 | 1000 | 0.3-0.6 | 0.3-0.6 |
| | Allele G | Allele A | Allele G | Allele A | Allele G | Allele A | Allele G | Allele A |
| Rs2734986 | 500 | 1000 | 0.9-1.0 | 0.9-1.0 | 400 | 700 | 0.2-0.5 | 0.4-0.8 |
| | Allele G | Allele A | Allele G | Allele A | Allele G | Allele A | Allele G | Allele A |
| Rs2844821 | 500 | 500 | 0.9-1.0 | 0.9-1.0 | 300 | 700 | 0.2-0.6 | 0.4-0.8 |
| | Allele A | Allele G | Allele A | Allele G | Allele A | Allele G | Allele A | Allele G |
| Rs2596477 | 500 | 500 | 0.9-1.0 | 0.9-1.0 | 300 | 300 | 0.25-0.6 | 0.24-0.6 |
| | Allele G | Allele A | Allele G | Allele A | Allele G | Allele A | Allele G | Allele A |
| Rs660895 | 1000 | 1000 | 0.9-1.00 | 0.9-1.0 | 500 | 500 | 0.25-0.75 | 0.25-0.75 |
| | Allele A | Allele G | Allele A | Allele G | Allele A | Allele G | Allele A | Allele G |
| Rs3094170 | 500 | 1000 | 0.9-1 | 0.9-1.0 | 500 | 1000 | 0.2-0.6 | 0.3-0.8 |
| | Allele A | Allele G | Allele A | Allele G | Allele A | Allele G | Allele A | Allele G |
| Rs3129860 | 1000 | 1000 | 0.9-1.0 | 0.9-1.0 | 1000 | 1000 | 0.30-0.75 | 0.30-0.75 |
| | Allele A | Allele G | Allele A | Allele G | Allele A | Allele G | Allele A | Allele G |
| Rs3130933 | 1000 | 1000 | 0.9-1.0 | 0.9-1.0 | 1000 | 1000 | 0.30-0.75 | 0.30-0.75 |

These cut-offs can be established as required according to the samples to be analysed.

### Example 5.1

### Homozygous Controls

A total of 59 samples, previously HLA genotyped by high resolution SBT and homozygous at all three HLA loci for each of the 3 common Caucasoid haplotypes, were used to determine cut-offs.

### Example 5.1.1

### HLA-A*0101-B*0801-DRB1*0301 Haplotype

### Example 5.1.1.1

### Rs2187668 MFI and Allelic Ratio Cut-Offs

MFI values for rs2187668 minor allele A were between 5000 and 8000 in the homozygous HLA-A*01-B*08-DRB1*03 samples while MFI values for major allele G were in the range of 300-600. MFI values for major allele G were slightly higher probably due to higher beads background for this particular SNP. These samples were considered negative for allele G because MFI values for allele A were 10 fold more than MFI values for allele G. Therefore samples were considered positive for minor allele A and negative for major allele G. Allelic ratios for minor allele A in homozygous controls were in the range of 0.87-0.94 and allelic ratios for major allele G were in the range of 0.05-0.15. Therefore MFI cut-offs for rs2187668 allele A in homozygous positive samples were determined to be above 2000 and below 700 for major allele G. Allelic ratio cut-off range for minor allele A was 0.80-1 and 0-0.20 for major allele G.

MFI values for minor allele A in the negative HLA-A*01-B*08-DRB1*03 samples were in the range of 200-400 and for major allele G in the range of 4000-7000. MFI values for major allele G were 10 times or more the MFI for minor allele A. Therefore samples were considered positive for allele G and negative for allele A. Cut-offs were determined to be as follows: MFI for major allele above 2000 and MFI minor allele bellow 500. Allelic ratio cut-offs: major allele Gin the range of 0.80-1 and minor allele A in the range of 0-0.20.

### Example 5.1.1.2

### Rs2734986 MFI and Allelic Ratio Cut-Offs

For HLA-A*01-B*08-DRB1*03 homozygous positive samples MFI values for minor allele G were in the range of 500-2000 while MFI values for major allele A were below 100. MFI values for the minor allele were at least 10 times higher than the MFI for major allele so the samples were considered positive for allele G and negative for allele A. Allelic ratios for minor allele G were above 0.9 while allelic ratios for major allele A were below 0.05. The selected cut-offs were as follows: minor allele G MFIs above 500 and at least 10 times more than major allele A; major allele A below 100 and at least 10 times less than MFI for minor allele G. Allelic ratios in the range of 0.80-1 for minor allele G and 0-0.10 for major allele A.

For homozygous negative samples MFIs for minor allele G were below 100 while major allele A were in the range of 1500-3500. MFIs for allele A were at least 10 times more than allele G so samples were considered positive for allele G and negative for allele A. Allelic ratios for allele A were an average of 0.99 and allele G below 0.05. Cut-off values determined for this SNP were: MFI values for allele A above 1000 and allele G below 100. Cut-offs values for the allelic ratios were: 0.0.80-1 allele A and 0-0.1 allele G.

### Example 5.1.2

### HLA-A*0201-B*4402-DRB1*0401

### Example 5.1.2.1

### Rs2844821 MFI and Allelic Ratio Cut-Offs

For HLA-A*02-B*44-DRB1*04 homozygous positive controls, MFI values for minor allele G were in the range of 700-1200 and MFI for major allele A were up to 120. Allelic ratios for minor allele G were between 0.87-0.98 while allelic ratios for major allele A were below 0.05. MFI value cut-offs for minor allele G were above 500 and at least 10 times higher than MFI values for major allele A. Allelic ratio cut-offs for minor allele G were between 0.80-1 and cut-offs for minor allele A were between 0-0.10.

In HLA-A*02-B*44-DRB1*04 negative samples, MFI values for minor allele G were below 50 and for major allele A were between 1000-2000. Allelic ratios for major allele A were above 0.9 while allelic ratios for minor allele G were below 0.1. Both MFI and allelic ratio values indicate that the controls were positive for major allele A and negative for minor allele G. MFI value cut-offs for major allele A were above 500 and at least 10 times higher than MFI values for minor allele G. Allelic ratio cut-offs determined for major allele A were between 0.8-1 and cut-offs for minor allele G between 0-0.10.

### Example 5.1.2.2

### Rs2596477 MFI and Allelic Ratios Cut-Offs

In HLA-A*02-B*44-DRB1*04 homozygous positive samples MFI values for the minor allele A were in the range of 1000-1500, 10 times more than MFI values for major allele G which were below 100. Allelic ratios for allele A were in the range of 0.95-1 while allelic ratios for allele G on the range of 0-0.05. MFI values cut-offs for minor allele A were above 500 and at least 10 times higher than major allele G MFI values.

In homozygous negative samples MFI values for major allele G were in the range 1000-1500, times higher than minor allele A MFI values which were below 100. Allelic ratios for major allele G were in the range of 0.95-1 and allelic ratios for minor allele A were an average of 0.05. MFI value cut-offs for the major allele G were above 500 and at least 10 times higher than MFI values for minor allele A.

### Example 5.1.2.3

### Rs660895 MFI and Allelic Ratio Cut-Offs

In HLA-A*02-B*44-DRB1*04 homozygous positive samples MFI values for minor allele G were in the range of 1500-2500, at least 10 times higher than the MFI values of up to 150 recorded for major allele A. Allelic ratios for minor allele G were in the range of 0.92-0.97 while allelic ratios for allele A were below 0.1.

Cut-offs MFI: allele G above 500 and at least 10 times higher than major allele A; allele A below 200 and 10 times less than minor allele G. Allelic ratios minor allele G 0.80-1; major allele A 0-0.20.

In homozygous negative samples MFI for minor allele G were in the range of 100-400 and MFI for major allele A were in the range of 2000-4000. MFI values for minor allele G were 10 times lower than MFIs for major allele A and therefore samples were considered negative for allele G. Allelic ratios for minor allele G were below 0.05 while allelic ratios for major allele A were above 0.9.

Cut-off: allele G below 500 and at least 10 times less than major allele A; allele A higher than 1500 and at least 10 times more than minor allele G.

### Example 5.1.3

### HLA-A*0301-B*0701-DRB1*1501

### Example 5.1.3.1

### Rs3094170 MFI and Allelic Ratio Cut-Offs

In HLA-A*03-B*07-DRB1*15 homozygous positive samples MFI values for minor allele A were in the range of 2000-4000 while MFIs for major allele G were below 100. Allelic ratios for the minor allele A were an average of 0.99 and allelic ratios for the major allele G were an average of 0.01. MFI value cut-off for minor allele A were above 500 and at least 10 times higher than MFI values for the major allele G. Allelic ratios cut-offs for minor allele A were between 0.85-1 and 0-0.10 for major allele G.

In HLA-A*03-B*07-DRB1*15 homozygous negative samples MFI values for the minor allele A were below 150 and 500-2000 for major allele G. Allelic ratios for the minor allele A were up to 0.1 while allelic ratios for the major allele G were above 0.9. MFI value cut-off for the major allele G were above 500 and at least 10 times higher than MFI values for the minor allele A.

### Example 5.1.3.2

### Rs3129860 MFI and Allelic Ratio Cut-Offs

In HLA-A*03-B*07-DRB1*15 homozygous positive samples MFI values for minor allele A were in the range of 3000-4500 while MFIs for major allele G were below 100. Allelic ratios for the minor allele A were an average of 0.99 and for the major allele G, were an average of 0.01. MFI value cut-off for minor allele A were above 500 and at least 10 times higher than MFI values for the major allele G. Allelic ratio cut-offs for minor allele A were between 0.8-1 and allelic ratio cut-offs for major allele G between 0-0.15.

In HLA-A*03-B*07-DRB1*15 homozygous negative samples MFI values for the minor allele A were below 50 and values for the major allele G were in the range of 4000-6000. Allelic ratios for the minor allele A were up to 0.01 while allelic ratios for the major allele G were an average of 0.99. MFI value cut-offs for the major allele G were above 500 and at least 10 times higher than MFI values for the minor allele A. Allelic ratio cut-offs for minor allele A were between 0.8-1 and allelic ratio cut-offs for major allele G between 0-0.15.

### Example 5.1.3.3

### Rs3130933 MFI and Allelic Ratio Cut-Offs

In HLA-A*03-B*07-DRB1*15 homozygous positive samples, MFI values for minor allele A were in the range of 3000-5000 while MFIs for major allele G were below 50. Allelic ratios for the minor allele A were an average of 0.99 and for the major allele G, they were an average of 0.01. MFI value cut-off for minor allele A was above 500 and at least 10 times higher than MFI values for the major allele G.

In HLA-A*03-B*07-DRB1*15 homozygous negative samples MFI values for the minor allele A were below 100 and values for the major allele G were in the range of 3000-5000. Allelic ratios for the minor allele A were an average of 0.01 while allelic ratios for the major allele G were an average of 0.99. MFI value cut-offs for the major allele G were above 500 and at least 10 times higher than MFI values for the minor allele A.

### Example 5.2

### Heterozygous Controls

The multiplex ASPE-Luminex reaction was validated using a panel of 71 heterozygous samples. MFI values generated for each individual tagSNPs were used to calculate allelic ratios in order to assign the genotype of the DNA samples.

### Example 5.2.1

### HLA-A*0101-B*0701-DRB1*0301

### Example 5.2.1.1

### Rs2187668 MFI and Allelic Ratio Cut-Offs Values Estimation

18 controls were heterozygous at both alleles for rs2187668. MFI values for minor allele A were in the range of 2500-5000 and a similar range was observed for major allele G also. The MFI values show the presence of both alleles of the SNP in these samples. These samples were considered positive for both alleles when were compared with controls where either allele A or allele G were absent. In samples that lack allele A or allele G, MFI values were in the range of 200-400 which is 10 times less than when either of these 2 alleles was present. Calculated allelic ratios based on the MFI values were in the range of 0.45-0.55. allelic ratio cut-offs for both alleles were determined to be between 0.3-0.7.

### Example 5.2.1.2

### Rs2734986 MFI and Allelic Ratio Cut-Offs Values Estimation

20 controls were heterozygous for rs2734986. MFI values for minor allele G were between 350 to 1500 and MFI values for major allele A were in the range of 1500-3000. These MFI values indicate the presence of both alleles in the heterozygous samples. However, for this SNP it is clear that major allele A hybridises better than allele G which results in higher MFIs values for allele A. Different hybridisation efficiencies for the 2 alleles of the SNP results in lower allelic ratios for allele G in the range of 0.2-0.4. These values were slightly lower than the arbitrary cut-offs suggested by Luminex. However, these values correspond to a heterozygous sample when compared with positive and negative samples for each of the SNP allele. In samples that lack allele G, MFI values were in the range of 1 to 50 which is 10 times less when compared with heterozygous sample MFIs of 300-1500. The same is observed when allelic ratios were calculated. Allelic ratio for allele G in heterozygous samples is between 0.2 and 0.40. Allelic ratios for samples lacking allele G were in the range of 0-0.01. Allelic ratios for allele A in the heterozygous controls were in the range of 0.5-0.8, while allelic ratios for samples lacking allele A were an average of 0.01. Therefore, based on these values, allelic ratios cut-offs for rs2734986 in heterozygous samples were in the lower range of 0.2-0.50 for minor allele G and 0.40-0.80 for major allele A. MFI values for allele G have to be at least 300 and at least 10 times higher than samples where allele G is not present. MFI values for allele A have to be at least 700 and 10 times more than samples lacking allele A.

### Example 5.2.2

### HLA-A*0201-B*4402-DRB1*0401

### Example 5.2.2.1

### Rs2596477 MFI and Allelic Ratio Cut-Offs

23 samples were heterozygous for rs2596477 with MFI values in the range of 200-1000 for both minor allele A and major allele G. In contrast with the other SNPs, lower MFI values were recorded for both alleles in heterozygous controls. However, for controls that lack either allele A or G, MFI values were below 50 and therefore 10 times less than MFI in the heterozygous controls. Calculated allelic ratios were an average of 0.4 which fall within the arbitrary Luminex cut-off for a heterozygous sample. MFI cut-offs were found to be above 200 for both alleles and allelic ratio cut-offs were in the range of 0.30-0.75.

### Example 5.2.2.2

### Rs2844821 MFI and Allelic Ratio Cut-Offs Estimation

23 samples were heterozygous for rs2844821 and express both alleles of the SNP. MFI values for minor allele G were in the range of 300-800 and for major allele A, in the range of 1000 to 2500. MFI values for both alleles indicate that allele A hybridises more efficiently than allele G which results in lower allelic ratios for allele G, in the range of 0.2-0.4. Allelic ratios for allele A were in the range of 0.60-0.80. These values correspond to heterozygous samples when they were compared with samples that lack either of the two alleles. In samples lacking allele G, MFI values recorded were in the range of 1-50, much lower than MFIs of 300-1000 in heterozygous controls expressing allele G. Allelic ratios for allele G in heterozygous controls were between 0.2-0.4 while in samples lacking allele G allelic ratios were an average of 0.01.

In samples where allele A was not expressed, recorded MFI values were an average of 50-100 and at least 10 times less than heterozygous controls where MFIs recorded were above 1000. Allelic ratios for allele A in heterozygous controls were between 0.6-0.8. In samples lacking allele A, allelic ratios were an average of 0.05. Allelic ratios for samples positive for allele A were an average of 0.98.

Therefore, allelic ratio cut-offs for SNP rs2844821 in heterozygous samples were established to be in the range of 0.20-0.60 for allele G and 0.40-0.80 for allele A. MFI values must be above 250 for allele G and at least 10 times higher than MFI values in samples lacking allele G. MFI values for allele A must be at least 500 and at least 10 times higher than samples where allele A is not present.

### Example 5.2.2.3

### Rs660895 MFI and Allelic Ratio Cut-Offs

24 heterozygous controls were tested for rs660895 expressing both alleles of the SNP. MFI values for the minor allele G in the heterozygous controls were between 1000-2500 and allelic ratios in the range of 0.30-0.70. MFI values for the major allele A in the heterozygous controls were between 1000-2500 and allelic ratios in the range of 0.30-0.70. MFI value cut off for both alleles in heterozygous controls were above 500 and at least 10 times more than samples lacking either of the 2 alleles.

### Example 5.2.3

### HLA-A*0301-B*0701-DRB1*1501

### Example 5.2.3.1

### Rs3094170 MFI and Allelic Ratio Cut-Offs

19 samples were heterozygous for rs3094170. MFI values for minor allele A were in the range of 300-1200 with resulting allelic ratios in the range of 0.20-0.50. MFI values for major allele G were in the range of 1500-2500 with resulting allelic ratios in the range of 0.50 to 0.80. The MFI and allelic ratios indicate that allele G hybridises more efficiently than allele A. These values indicate that both alleles of the SNP were present when compared with controls lacking either of the two alleles. For samples lacking either allele, MFI values were below 50 and resulting allelic ratios were an average of 0.01. Samples expressing either of the 2 allele have an average allelic ratio of 0.99.

### Example 5.2.3.2

### Rs3129860 MFI and Allelic Ratio Cut-Offs

17 samples were heterozygous for rs3129860. MFI values for both alleles A and G were in the range of 2000-3500. Allelic ratios for both alleles were an average of 0.45-0.55.

### Example 5.2.3.3

### Rs3130933 MFI and Allelic Ratio Cut-Offs

17 samples were heterozygous for rs3130933. Both A and G alleles were present in these controls and both have MFI values in the range of 1500-3500. Allelic ratios were an average of 0.55 which fall within the arbitrary Luminex cut-offs for heterozygous samples.

### Example 6

### HLA Haplotyping of a Blind Cohort of Donor Samples Using the Multiplex ASPE-Luminex Assay

94 blind DNA samples already HLA typed were tested using the validated ASPE-Luminex assay and genotypes were assigned based on the MFI and allelic ratio cut-off determined for each individual SNP. 100% correlation was found for HLA-A*0101-B*0801-DRB1*0301 and HLA-A*0301-B*0701-DRB1*1501 in both homozygous and heterozygous samples. 100% correlation was found also for HLA-A*0201-B*4402-DRB1*0401 homozygous samples. Rs660895 which tags HLA-DRB1*0401 allele also tags HLA-DRB1*08. Therefore 5% from the total samples was wrongly assigned HLA-DRB1*0401 instead of HLA-DRB1*08.1% of the samples were wrongly assigned HLA-A*0201-B*4402-DRB1*0401 heterozygous.

Allele and genotype frequencies for each individual SNP were calculated using www.geneva.unige.ch/generate/statistical tool for HLA population data analysis (Table 8). SNP allele and genotype frequencies were concordant with previous data published by HapMap Consortium for people with northern and Western European ancestry (CEU) population.

TABLE 8
Genotype and allele frequencies and p values for Hardy-Weinberg
equilibrium (HWE) in samples of
94 unrelated North European Caucasoid subjects

| | | | | | P value |
|---|---|---|---|---|---|
| | | | | | for |
| SNP ID | Chromosome | HLA allele | Allele frequency | | HWE |
| Rs2734986 | 6 | HLA-A*0101 | G: 0.22 | A: 0.77 | 0.01528 |
| Rs2187669 | 6 | HLA- | A: 0.19 | G: 0.80 | 0.31794 |
| | | DRB1*0301 | | | |
| Rs2844821 | 6 | HLA-A*0201 | G: 0.26 | A: 0.73 | 0.28230 |
| Rs2596477 | 6 | HLA-B*4401 | A: 0.16 | G: 0.83 | 0.11284 |
| Rs660905 | 6 | HLA- | G: 0.14 | A: 0.86 | 0.20003 |
| | | DRB1*0401 | | | |
| Rs3094170 | 6 | HLA-A*0301 | A: 0.13 | G: 0.86 | 0.19875 |
| Rs3129860 | 6 | HLA-B*0701 | A: 0.14 | G: 0.85 | 0.11132 |
| Rs3130933 | 6 | HLA- | A: 0.13 | G: 0.86 | 0.68712 |
| | | DRB1*1501 | | | |

Deviation from the Hardy-Weinberg equilibrium was calculated using Arlequin software. Apart from rs2734986 with a p value of 0.01, no deviation from Hardy-Weinberg equilibrium was observed in any other locus (p>0.05).

### REFERENCES

Ally T A, Baschal M M, Jahromi M S, Fernando S R, Babu S R et at (2008) Analysis of single nucleotide polymorphisms identifies major type 1A diabetes locus telomeric of the major histocompatibility complex. Diabetes, 57: 770-776
Bashal E E, Aly T A, Jasinki J M, Steck A K, Noble J A et at (2008) Defining multiple common “completely” conserved major histocompatibility complex SNP haplotype. Clinical Immunology, 132: 203-214
Carrington M, Stephens J C, Klitz W, Begovich A B, Erlich H A & Mann D (1994). Major histocompatibility complex class II haplotypes and linkage disequilibrium values observed in the CEPH families. Hum. Immunol., 41: 234-240
Dawkins R, Leelayuwat C, Gaudieri S, Tay G, Hui J et at (1999) Genomics of the major histocompatibility complex: haplotypes, duplication, retroviruses and disease. Immunol. Rev, 167: 275-304.
Degli-Esposti M A, Leaver A L, Christiansen F T, Witt C S, Abraham L J & Dawkins R L (1992) Ancestral haplotypes: Conserved Population MEW Haplotypes. Hum Immunol, 34: 242-252
DeBakker P I W et at (2006a) Transferability of tagSNPs in genetic association studies in multiple population. Nat Genet, 38: 1298-1303
DeBakker P I W, McVean G, Sabeti P C, Miretti M M, Green T, et at (2006b) A high-resolution HLA and SNP haplotype map for disease association studies in the extended human MEW. Nature Genetics, 38: 1166-1172
Dorak M T, Shao W, Machulla H K, Lobashevsky E S, Tang J, et at (2006) 1. Conserved extended haplotypes of the major histocompatibility complex: further characterization. Genes Immun, 7:450-67
Horton R, Wilming L, Rand V, Lovering R C, Bruford E A et al (2004). Gene map of the extended human MEW. Nature genetics, 5: 889-899
Horton R, Gibson R, Coggill P, Miretti N, Allcock R J et at (2008) Variation analysis and gene annotation of eight MHC haplotypes: The MHC Haplotype Project. Immunogenetics, 60 (1): 1-18
Leslie, S, Donelly P & McVean G (2008) A statistical method for predicting classical HLA alleles from SNP data. Am J Hum Genet, 82, 48-56.
Miretti M M, Walsh E C, Ke X, Delgado M, Griffiths M et at (2005) A high resolution linkage disequilibrium map of the human major histocompatibility complex and first generation of tag single nucleotide polymorphisms. Am J Hum Genet, 76:634-646.
Shiina T, Hosomich K, Inoko H & Kulski J K (2009) The HLA genomic loci map: expression, interaction, diversity and disease. Journal of Human Genetics, 54: 15-39
Smith W P, Vu Q, Li S S, Hansen J A, Zhao L P, Geraghty D E (2006). Toward understanding MHC disease associations: partial resequencing of 46 distinct HLA haplotypes. Genomics, 87:561-9
Stewart C A, Horton R, Allcock R J, Ashurst J L, Atrazhev A M et al (2004) Complete MEW haplotype sequencing for common disease gene mapping. Genome Res 14:1176-1187
Stephens M., Smith N J. And Donelly P. (2001) A new statistical method for haplotype reconstruction from population data. Am. J. Hum. genet. 68:978-989
The International Hap Map Consortium (2003) The international Hap Map project. Nature, 426: 789-794
The International HapMap Consortium (2004) Integrating ethics and science in the international HapMap project, Nat. rev. Genet, 5: 467-475
The International HapMap Consortium (2005) A haplotype map of the human genome. Nature, 437: 1299-1320
The International HapMap Consortium (2007) A second generation human haplotype map of over 3.1 million SNPs. Nature, 449: 851-861
The International HapMap3 Consortium (2010) integrating common and rare genetic variation in diverse human population. Nature, 467: 52-58
The International Human Genome sequencing consortium (2004) Finishing the euchromatic sequence of the human genome. Nature, 431: 931-945

1. A method for genotyping a human subject comprising the steps of isolating a nucleic acid from a sample from the subject, and identifying a selection of tag single nucleotide polymorphisms (tagSNPs) in a selection of human leukocyte antigen (HLA) haplotypes.

2. The method according to claim 1, wherein the HLA haplotypes or the tagSNPs are in chromosome 6.

3. The method according to claim 1, wherein the selection of HLA haplotypes is HLA-A*01-B*08-DRB1*03, HLA-A*02-B*44-DRB1*04 and HLA-A*03-B*07-DRB1*15.

4. The method according to claim 1, wherein the selection of tagSNPs is Rs2734986, Rs2187668, Rs2844821, Rs2596477, Rs660895, Rs3094170, Rs3130933 and Rs3129860.

5. The method according to claim 4, wherein Rs2734986 and Rs2187668 are for HLA-A*01-B*08-DRB1*0.

6. The method according to claim 4, wherein Rs2844821, Rs2596477 and Rs660895 are for HLA-A*02-B*44-DRB1*04.

7. The method according to claim 4, wherein Rs3094170, Rs3130933 and Rs3129860 are for HLA-A*03-B*07-DRB1*15.

8. The method according to claim 1, wherein the tagSNPs are identified using the primer sequences in SEQ ID NO. 1 to 34.

9. A method of screening samples for a genotype using the method as defined in claim 1.

10. The method of claim 9, which is a high-through-put method.

11. A method of matching donor and recipient based on the genotype determined by the method of claim 1.

12. A method of HLA haplotyping a human subject according to the method of claim 1.

13. A method of determining migration of HLA haplotypes in a human population using the method according to claim 1.

14. A nucleotide sequence according to any one of SEQ ID NO. 1 to 34.

15. A method of identifying tagSNPs in a sample comprising amplifying a nucleic acid from the sample using a selection of nucleotide primers having the sequences according to claim 14.

16. A kit for HLA haplotyping a human subject comprising tagSNP primers and allele specific primer extension (ASPE) primers.

17. A kit according to claim 16 wherein the tagSNP primers are selected from SEQ ID NO. 1 to 16.

18. The kit according to claim 16, or 17 wherein the ASPE primers are selected from SEQ ID NO. 17 to 32.


SEQUENCE LISTING (66 sequences)

SEQ ID NO: 1 - DNA, 23 bp, Artificial Sequence
  Primer
tgcctttctagggagcaaccact

SEQ ID NO: 2 - DNA, 20 bp, Artificial Sequence
  Primer
tggctccgcttggacctttg

SEQ ID NO: 3 - DNA, 23 bp, Artificial Sequence
  Primer
gtgaggtgacacatatgaggcag

SEQ ID NO: 4 - DNA, 25 bp, Artificial Sequence
  Primer
ggctgaatgccttcaacaatcattt

SEQ ID NO: 5 - DNA, 21 bp, Artificial Sequence
  Primer
tgcacagagtgaagccaccca

SEQ ID NO: 6 - DNA, 20 bp, Artificial Sequence
  Primer
actgcctgcgggtactgcct

SEQ ID NO: 7 - DNA, 22 bp, Artificial Sequence
  Primer
taactcctacttggccagacct

SEQ ID NO: 8 - DNA, 21 bp, Artificial Sequence
  Primer
tgctcagatgcccacagcact

SEQ ID NO: 9 - DNA, 22 bp, Artificial Sequence
  Primer
ataactgcttacaagtgtggcc

SEQ ID NO: 10 - DNA, 26 bp, Artificial Sequence
  Primer
gtagtaagtcacttagatgaacatga

SEQ ID NO: 11 - DNA, 20 bp, Artificial Sequence
  Primer
agagcatttgccgaggccga

SEQ ID NO: 12 - DNA, 21 bp, Artificial Sequence
  Primer
gtccagctcctgggtcctccc

SEQ ID NO: 13 - DNA, 27 bp, Artificial Sequence
  Primer
tctcatcttcacactccttgtcttcct

SEQ ID NO: 14 - DNA, 24 bp, Artificial Sequence
  Primer
acgacagtcatttctgccaccttt

SEQ ID NO: 15 - DNA, 20 bp, Artificial Sequence
  Primer
gggtgggggcaggggagttt

SEQ ID NO: 16 - DNA, 20 bp, Artificial Sequence
  Primer
ggtgcaatcccagctctccc

SEQ ID NO: 17 - DNA, 24 bp, Artificial Sequence
  Primer
acatatgaggcagctgagagtaaa

SEQ ID NO: 18 - DNA, 25 bp, Artificial Sequence
  Primer
cacatatgaggcagctgagagtaag

SEQ ID NO: 19 - DNA, 23 bp, Artificial Sequence
  Primer
agcaccaaagcaccatttcttta

SEQ ID NO: 20 - DNA, 25 bp, Artificial Sequence
  Primer
taagcaccaaagcaccatttctttg

SEQ ID NO: 21 - DNA, 23 bp, Artificial Sequence
  Primer
ccaacaaaaacaagacttgtatg

SEQ ID NO: 22 - DNA, 23 bp, Artificial Sequence
  Primer
ccaacaaaaacaagacttgtata

SEQ ID NO: 23 - DNA, 25 bp, Artificial Sequence
  Primer
cttagcagtgcatcagtgtcaattg

SEQ ID NO: 24 - DNA, 25 bp, Artificial Sequence
  Primer
cttagcagtgcatcagtgtcaatta

SEQ ID NO: 25 - DNA, 27 bp, Artificial Sequence
  Primer
ttcctattttttccatattcttgacaa

SEQ ID NO: 26 - DNA, 27 bp, Artificial Sequence
  Primer
ttcctattttttccatattcttgacag

SEQ ID NO: 27 - DNA, 25 bp, Artificial Sequence
  Primer
caccccaaggctctctcccattagg

SEQ ID NO: 28 - DNA, 25 bp, Artificial Sequence
  Primer
caccccaaggctctctcccattaga

SEQ ID NO: 29 - DNA, 26 bp, Artificial Sequence
  Primer
gctaaccatgtaccttaaataaacca

SEQ ID NO: 30 - DNA, 26 bp, Artificial Sequence
  Primer
gctaaccatgtaccttaaataaaccg

SEQ ID NO: 31 - DNA, 24 bp, Artificial Sequence
  Primer
gtttgtccttcagttactgaggta

SEQ ID NO: 32 - DNA, 22 bp, Artificial Sequence
  Primer
ttgtccttcagttactgaggtg

SEQ ID NO: 33 - DNA, 22 bp, Artificial Sequence
  Arificial Sequence
atatgaggcagctgagagtaag

SEQ ID NO: 34 - DNA, 24 bp, Artificial Sequence
  Artificial sequence
ttagcagtgcatcagtgtcaattg

SEQ ID NO: 35 - DNA, 24 bp, Artificial Sequence
  TAG nucleotide sequence
tcaacaatcttttacaatcaaatc

SEQ ID NO: 36 - DNA, 24 bp, Artificial Sequence
  Anti-TAG nucleotide sequence
gatttgattgtaaaagattgttga

SEQ ID NO: 37 - DNA, 24 bp, Artificial Sequence
  TAG nucleotide sequence
ctaaatacttcacaattcatctaa

SEQ ID NO: 38 - DNA, 24 bp, Artificial Sequence
  Anti-TAG nucleotide sequence
ttagatgaattgtgaagtatttag

SEQ ID NO: 39 - DNA, 24 bp, Artificial Sequence
  TAG nucleotide sequence
tcatttcaatcaatcatcaacaat

SEQ ID NO: 40 - DNA, 24 bp, Artificial Sequence
  Anti-TAG nucleotide sequence
attgttgatgattgattgaaatga

SEQ ID NO: 41 - DNA, 24 bp, Artificial Sequence
  TAG nucleotide sequence
tcaatcaattacttactcaaatac

SEQ ID NO: 42 - DNA, 24 bp, Artificial Sequence
  Anti-TAG nucleotide sequence
gtatttgagtaagtaattgattga

SEQ ID NO: 43 - DNA, 24 bp, Artificial Sequence
  TAG nucleotide sequence
ttacctttatacctttctttttac

SEQ ID NO: 44 - DNA, 24 bp, Artificial Sequence
  Anti-TAG nucleotide sequence
gtaaaaagaaaggtataaaggtaa

SEQ ID NO: 45 - DNA, 24 bp, Artificial Sequence
  TAG nucleotide sequence
tcaattacttcactttaatccttt

SEQ ID NO: 46 - DNA, 24 bp, Artificial Sequence
  Anti-TAG nucleotide sequence
aaaggattaaagtgaagtaattga

SEQ ID NO: 47 - DNA, 24 bp, Artificial Sequence
  TAG nucleotide sequence
tacactttctttctttctttcttt

SEQ ID NO: 48 - DNA, 24 bp, Artificial Sequence
  Anti-TAG nucleotide sequence
aaagaaagaaagaaagaaagtgta

SEQ ID NO: 49 - DNA, 24 bp, Artificial Sequence
  TAG nucleotide sequence
tcataatctcaacaatctttcttt

SEQ ID NO: 50 - DNA, 24 bp, Artificial Sequence
  Anti-TAG nucleotide sequence
aaagaaagattgttgagattatga

SEQ ID NO: 51 - DNA, 24 bp, Artificial Sequence
  TAG nucleotide sequence
aatccttttacattcattacttac

SEQ ID NO: 52 - DNA, 24 bp, Artificial Sequence
  Anti-TAG nucleotide sequence
gtaagtaatgaatgtaaaaggatt

SEQ ID NO: 53 - DNA, 24 bp, Artificial Sequence
  TAG nucleotide sequence
ttacttcactttctatttacaatc

SEQ ID NO: 54 - DNA, 24 bp, Artificial Sequence
  Anti-TAG nucleotide sequence
gattgtaaatagaaagtgaagtaa

SEQ ID NO: 55 - DNA, 24 bp, Artificial Sequence
  TAG nucleotide sequence
ctacaaacaaacaaacattatcaa

SEQ ID NO: 56 - DNA, 24 bp, Artificial Sequence
  Anti-TAG nucleotide sequence
ttgataatgtttgtttgtttgtag

SEQ ID NO: 57 - DNA, 24 bp, Artificial Sequence
  TAG nucleotide sequence
aatcttactacaaatcctttcttt

SEQ ID NO: 58 - DNA, 24 bp, Artificial Sequence
  Anti-TAG nucleotide sequence
aaagaaaggatttgtagtaagatt

SEQ ID NO: 59 - DNA, 24 bp, Artificial Sequence
  TAG nucleotide sequence
tcatcaatcaatctttttcacttt

SEQ ID NO: 60 - DNA, 24 bp, Artificial Sequence
  Anti-TAG nucleotide sequence
aaagtgaaaaagattgattgatga

SEQ ID NO: 61 - DNA, 24 bp, Artificial Sequence
  TAG nucleotide sequence
tcaaaatctcaaatactcaaatca

SEQ ID NO: 62 - DNA, 24 bp, Artificial Sequence
  Anti-TAG nucleotide sequence
tgatttgagtatttgagattttga

SEQ ID NO: 63 - DNA, 24 bp, Artificial Sequence
  TAG nucleotide sequence
aatctaacaaactcatctaaatac

SEQ ID NO: 64 - DNA, 24 bp, Artificial Sequence
  Anti-TAG nucleotide sequence
gtatttagatgagtttgttagatt

SEQ ID NO: 65 - DNA, 24 bp, Artificial Sequence
  TAG nucleotide sequence
caattaactacatacaatacatac

SEQ ID NO: 66 - DNA, 24 bp, Artificial Sequence
  Anti-TAG nucleotide sequence
gtatgtattgtatgtagttaattg